(19) United States
(12) Patent Application Publication
(10) Pub. No.: US 2018/0242863 A1
(43) Pub. Date: Aug. 30, 2018

(54) WEARABLE DEVICE FOR ASSESSING THE LIKELIHOOD OF THE ONSET OF CARDIAC ARREST AND A METHOD THEREO

(71) Applicant: Heartisans Limited, Hong Kong (CN)

(72) Inventor: Hin Wai LUI, Hong Kong (CN)

(73) Assignee: Heartisans Limited, Hong Kong (CN)

(21) Appl. No.: 15/553,894

(22) PCT Filed: Jan. 8, 2016
(86) PCT No.: PCT/CN2016/070471

§ 371 Date: Aug. 25, 2017

(51) Int. Cl. A61B 5/024; A61B 5/00

(52) U.S. Cl. CPC A61B 5/02438; A61B 5/02416; A61B 5/681; A61B 5/7267; A61B 5/746; A61B 2562/0219; A61B 5/721; A61B 5/6843; A61B 5/7275; A61B 5/7455; A61B 5/02405

(57) ABSTRACT

A device and a method for assessing the likelihood of an imminent occurrence of cardiac arrest. The device comprises an optical sensor for monitoring the heart rhythm of a person. A Machine Learning Algorithm such as the Artificial neural network (ANN) algorithm analyse features from a trending of pulse intervals in the person's heart rhythm in real time to make the assessment. The device is provided in wearable form, such as a wrist worn device.

### FIELD OF THE INVENTION

The current invention relates to devices and methods for assessing the risk of occurrence of cardiac arrests of persons, and for giving advance warning.

### BACKGROUND

Cardiovascular diseases account for 17.3 million deaths in 2013, representing 30% of all global deaths, according to the World Health Organisation. This is greater than any other cause of death.

Of different cardiovascular diseases, cardiac arrest is increasingly the more common cause of sudden deaths. However, statistics has shown that survival rate may be as high as 30% if a person receives defibrillation or cardiopulmonary resuscitation (CPR) within 3 to 5 minutes of an onset of cardiac arrest. On the other hand, survival rate decreases by 7 to 10% for every minute that treatment is delayed. Therefore, it is in the interest of people if doctors were able to predict whether cardiac arrest is likely to occur soon. Unfortunately, the only way doctors may attempt at predicting cardiac arrest for any person is to rely on reading indirect indicators, such as his cholesterol level, family illness history, any recent heart pain and so on. While these indicators may tell whether a person is a candidate of cardiac arrest, they provide no clue to the moment cardiac arrest may occur.

When a person suffers cardiac arrest in the absence of human company and if his movements were impaired the cardiac arrest, he might not be able to call for help or contact emergency services and end up receiving late or no treatment. This is why it is not uncommon to hear of the lives of elderly people living in single occupant flats claimed by cardiac arrest.

It is possible to monitor a person potentially at risk of cardiac arrest by making him stay in a hospital or a care-giving home, where a round the clock watch may be imposed. However, such a plan demands huge financial resource and takes up valuable hospital beds. Furthermore, there is a possibility that cardiac arrest may never strike and the person may well enjoy a lifetime of normal work and leisure. Therefore, it is not practical to require a person to live under constant watch only to ensure that help is at hand readily.

Electrocardiography is the most common method of assessing heart condition. An electrocardiogram (ECG) device obtains electrical signals of the sinoatrial node of the heart. However, interpretation of the ECG requires significant training. To take an ECG, electrodes of an electrocardiogram device must be placed on specific points of the chest or other parts of the body in such a way that at least two electrical contacts form a complete circuit across the heart. The taking and interpreting of an ECG is not easily conducted in a domestic setting, where trained personnel is usually not available. Furthermore, the typical way in which an ECG is interpreted does not provide fool-proof detection of cardiac arrest, as ECG indicators of cardiac arrest might not be present all the time. It is not unheard of that a patient has been sent home by hospital personnel on observing normal ECG only to suffer cardiac arrest while on the way.

U.S. Pat. No. 9,161,705 proposed a wearable ECG monitor which can tell from the morphology of an ECG whether the wearer is about to suffer heart attack. ‘Heart attack’ refers to the case of a coronary artery being blocked resulting in lack of oxygen to the heart itself, whereas ‘cardiac arrest’ refers to heart arrhythmia that results in no blood being pumped by the heart. This ECG monitor is worn on a belt around the wearer's chest and has to be used with a smart phone application. However, it is uncomfortable for any person to be wearing a chest belt for an extensive period of time daily. Moreover, the position of the chest belt will tend to run as the person engages himself in daily activities, such that electrical signals cannot be harvested properly for accurate interpretation of heart condition.

The FDA has approved for use a device called AliveCor Heart Monitor, which is an ECG recorder attached to a mobile device. Its users would open an application in the mobile device and place their fingers on sensors provided on the ECG recorder to have their ECG recorded. Subsequently, the users would be able to collect, view, save and send the ECG to their personal cardiologist or to AliveCor's registered cardiologists for consultation. However, the users would only be able to monitor and record heart rhythms for the instant when the mobile application is open. Long term continuous tracking of heart condition is not possible.

None of the proposed solutions allow a person to be monitored around the clock in a practical and effective manner. Furthermore, none of the proposed solutions is able to provide any useful warning of imminent cardiac arrest before it occurs.

Accordingly, it is desirable to propose a method or device which could provide a possibility of warning of imminent onset of cardiac arrest, and to provide so continuously and around the clock.

### STATEMENTS OF INVENTION

In a first aspect, the invention proposes a wearable device suitable for assessing the likelihood of onset of cardiac arrest, comprising: a wearable configuration for being worn by a body part, a light source configured for illuminating the body part, an optical sensor configured to detect light rebounded from the body part, wherein the heart rhythm of a person wearing the wearable device is detected by the optical sensor from the pulsation in intensity of the rebounded light, and the wearable device is capable of subjecting the heart rhythm to analysis and issuing an alarm if the heart rhythm comprises patterns pre-determined as preceding onset of cardiac arrest.

Advantageously, the proposed invention provides the possibility that the heart condition of a person may be monitored by a technology which is rugged and robust enough for use in a daily, round the clock deployment. In contrast to an ECG monitor, a light based detection system does not require two points of electrical contacts to form a complete circuit across the heart and may hence be made smaller and to be worn on any part of the body, such as the wrist.

Preferably, the proposed wearable device is capable of subjecting heart rhythm to analysis by a machine learning algorithm, such as an artificial neural network, for assessing the risk of the onset of cardiac arrest for advance warning. Typically, the analysis by the machine learning algorithm is made on extracting features of heart rate variation observed from the heart rhythm.

Heart rate variation refers to the variation of the interval between pulses or heart beats. Optionally, any other aspects of the heart rhythm may be analysed instead, such as pulse intensity instead of the interval between pulses.

Advantageously, use of artificial neural network allows modelling to be made of multiple variables for predicting an outcome; many features of heart rhythm may be considered at once for assessing the risk of the onset of cardiac arrest for advance warning. Furthermore, a trained artificial neural network can be improved or re-trained indefinitely with more user data as the wearable device is used by more people.

Optionally, the artificial neural network is trained using records of heart rhythm of at least the 15 minutes leading up to onset of cardiac arrest. Alternatively, the artificial neural network is trained using records of heart rhythm of at least the 30 minutes leading up to onset of cardiac arrest. This provides a possibility of training the artificial neural network to determine if cardiac arrest is likely to occur with a 15 or 30-minute lead time, improving the chance of the person finding help in time.

Preferably, the wearable device further comprises an accelerometer configured to detect movements of the wearer, wherein the analysis of the heart rhythm include cancellation of the effects from movements of the wearer on the heart rhythm detected by the optical sensor.

Preferably, the wearable device is configured as a wristband, as the wrist is a convenient location on the body for 24 hour, daily wearing of the device.

Preferably, the wearable device further comprises a skin impedance sensor, the skin impedance sensor positioned on the wearable device such that impedance measured by the skin impedance sensor is indicative of tight or sufficient contact between the optical sensor and the skin of the wearer.

In a second aspect, the invention proposes a method for assessing the risk of the onset of cardiac arrest for advance warning, comprising the steps of: providing a light source for illuminating a body part of a person, detecting from the pulsation in intensity of the rebounded light rebounded from the body part to detect the heart rhythm of the person, subjecting the heart rhythm to analysis, and raising an alarm if the analysis determines that the heart rhythm comprises patterns pre-determined as preceding onset of cardiac arrest.

Preferably, the step of subjecting the heart rhythm to analysis is to apply an algorithm to analyse the heart rhythm, and the algorithm is a machine learning algorithm.

Preferably, the machine learning algorithm is an artificial neural network.

Preferably, the analysis by the machine learning algorithm is made on heart rate variation observed from the heart rhythm.

Preferably, the heart rate variation is the variation in intervals between a pre-determined number of heat beats, such as any two heart beats or pulses.

Optionally, any other aspects of the heart rhythm may be analysed instead, such as pulse intensity instead of the interval between pulses.

Preferably, the heart rhythm is observed in a number of windows of time, each window providing a period of heart rhythm to be subjected to concurrent analysis with the periods of heart rhythm observed in the other windows, and the period of heart rhythm observed in each window of time being a period of the heart rhythm as recorded historically or currently observed. Typically, the windows of time do not overlap. Using different, non-overlapping windows of heart rhythm will increase the number of observations to be fed into the artificial neural network at any instant in time, which gives rise to better accuracy in determining the likelihood of a cardiac arrest. It is preferable that three windows are used.

Typically, the machine learning algorithm is capable of being re-trained using the heart rhythm of a person who suffers cardiac arrest when being monitored. This makes possible an advantage that embodiments of the invention get better at predicting cardiac arrest as they are used and as more data is provided to update or re-train the embodiments.

Typically, the artificial neural network is trained using records of heart rhythm of at least the 15 minutes leading up to cardiac arrest. More preferably, the artificial neural network is trained using records of heart rhythm of at least the 30 minutes leading up to cardiac arrest. Currently, readily available records of heart rhythm preceding cardiac arrest are only of the 5 to 15 minutes prior to onset. However, the proposed method and device provide a possibility of continuous monitoring of people. If anyone suffers cardiac arrest when monitored by the proposed method or the device, records of heart rhythm of at least the 30 minutes, or even 60 minutes, leading up to cardiac arrest, will be made available. These records can be used to re-train the artificial neural network to recognise patterns pre-determined as preceding onset of cardiac arrest by 30 minutes, or even 60 minutes.

### BRIEF DESCRIPTION OF DRAWINGS

It will be convenient to further describe the present invention with respect to the accompanying drawings that illustrate possible arrangements of the invention, in which like integers refer to like parts. Other embodiments of the invention are possible, and consequently the particularity of the accompanying drawings is not to be understood as superseding the generality of the preceding description of the invention.

FIG. 1 shows an embodiment of the invention;

FIG. 2 shows the underside of the embodiment of FIG. 1;

FIG. 3 shows a scheme of the internal structure of the embodiment of FIG. 1;

FIG. 4 shows the embodiment of FIG. 1 in use by a person;

FIG. 5 illustrates the embodiment of FIG. 1 in a larger setting;

FIG. 6 shows a heart rhythm used in the embodiment of FIG. 1;

FIG. 7 shows a heart rhythm used in the embodiment of FIG. 1;

FIG. 8 shows a heart rhythm monitored in the embodiment of FIG. 1;

FIG. 9 shows a heart rhythm monitored in the embodiment of FIG. 1;

FIG. 10 illustrates an artificial neural network topography which can be used in the embodiment of FIG. 1;

FIG. 11 the embodiment of FIG. 1 in use;

FIG. 12 the embodiment of FIG. 1 in use; and

FIG. 13 is a flowchart explaining the embodiment of FIG. 1.

### DESCRIPTION OF PREFERRED EMBODIMENTS

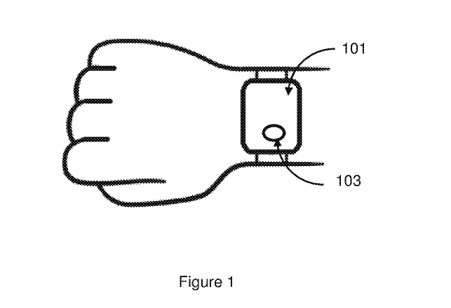

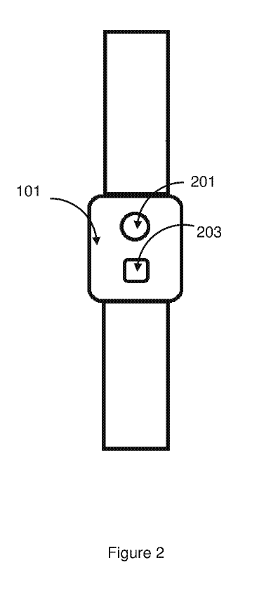

FIG. 1 shows a wrist wearable heart monitor 101 strapped to the wrist of a person. FIG. 2 provides a view of the underside of the heart monitor 101. On the underside of the heart monitor 101 is a PPG (photoplethysmocharty) sensor. A PPG sensor uses light-based technology to sense the rate of blood flow as controlled by the heart's pumping action. As a simplified description, PPG sensor comprises at least one light source 201 such as an LED (light emitting diode) and one corresponding optical sensor 203.

The heart monitor 101 is designed to be worn such that the light source 201 and the optical sensor 203 are placed somewhat snugly against the skin, in order to prevent ambient light from causing too much noise signals in the optical sensor 203.

In use, the light source 201 transmits light onto the person's skin, and the light is diffused and reflected by the surface of the skin and detected by the optical sensor 203. ‘Reflection’ is taken here to include the case wherein light penetrates beneath the skin surface but are diffused or rebounded back by the top layers of skin and tissue towards the optical sensor 203. The reflected or rebounded light will have a varying intensity which fluctuates in accordance with the pulsation of blood flow in the person's skin. In this way, the optical sensor 203 is able to detect the heart rhythm of the person.

A PPG sensor is small and only requires a single point of contact on a person's body, as oppose to a need of multiple points of contact for taking an ECG. Therefore, using a PPG sensor allows a small heart-monitoring device to be made in a convenient and portable form, such as the wrist worn configuration shown in FIG. 1, to be deployed on and worn by a person around the clock daily.

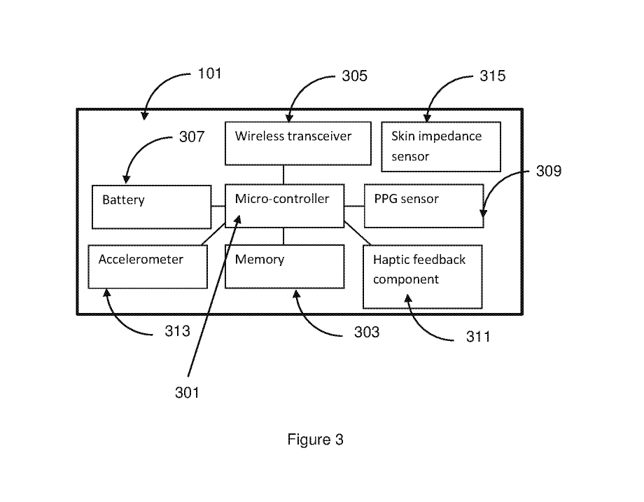

FIG. 3 is a schematic diagram of one possible internal structure of the heart monitor 101. The heart monitor 101 comprises a microcontroller 301 and a memory 303. The microcontroller 301 operates the optical sensor 203 to detect light reflected from the person's skin.

The memory 303 contains an algorithm for assessing the heart rhythm of the person. Preferably, the memory 303 has capacity to store at least a one month history of the person's heart rhythm.

A wireless transceiver 305 is provided for wireless communication with a mobile phone or any other device requiring information from the heart monitor 101. The wireless communication protocol for communicating with a mobile phone or a computer is preferably Bluetooth Low Energy. To operate Bluetooth communication, the top face of the heart monitor 101 as shown in FIG. 1 comprises a button 103 for initiating Bluetooth synchronisation with, for example, a mobile phone application.

Optionally, the heart monitor 101 comprises a haptic feedback component 311 for issuing an alarm to the person wearing it. Alternatively, the alarm may be replaced by or may include an audio alarm such as a small siren, or a visual alarm such as a blinking LED.

A replaceable and rechargeable battery 307 is provided to supply power to all the components in the heart monitor 101. The battery 307 is preferably a rechargeable and replaceable one, as it allows the person to swap the battery 307 quickly at any time, such that there is no need to wait for the battery 307 to charge up. This provides the advantage that the person may benefit from almost seamless, continual monitoring of his heart.

In use, the PPG samples the person's heart rhythm in real time while the microcontroller 301 analyses the heart rhythm by applying the algorithm. The algorithm calculates from the heart rhythm the likelihood of an onset of cardiac arrest in the near future. If the heart monitor 101 detects from the heart rhythm of the person that cardiac arrest is likely to occur, it raises an alarm.

Furthermore, the heart monitor 101 optionally comprises a skin impedance sensor 315 (not illustrated in FIG. 1) positioned on the underside of the heart monitor 101, adjacent the light source 201 and the optical sensor 203. A skin impedance sensor 315 measures impedance or conductance of skin surface. Impedance of skin is different from that of air. Therefore, if the skin impedance sensor 315 is in contact with the skin of the person, certain impedance should be measured. This implies that the optical sensor 203 is placed in tight contact or sufficient contact with the skin, reducing the possibility of ambient light affecting the reading of the optical sensor 203. If there is a small gap between the person's skin and the optical sensor 203, there will also be a small gap between the skin impedance sensor 315 and the skin, and the skin impedance sensor 315 will not detect impedance typical of skin but will detect impedance somewhat typical of air. In this way, the skin impedance sensor 315 is useable to determine whether the light source 201 and the optical sensor 203 have been placed in sufficient contact with the skin in order for the PPG sensor 309 to read heart rhythm properly. Preferably, the heart monitor 101 is able to alert the person that the light source 201 and the optical sensor 203 are not placed tightly enough against the skin, such as by issuing a series of haptic signals in a specific rhythm. Furthermore, if the skin impedance sensor 315 determines that the light source 201 and the optical sensor 203 are not in contact with the skin, data read by the optical sensor 203 is rejected and not taken to assess the likelihood of onset of cardiac arrest.

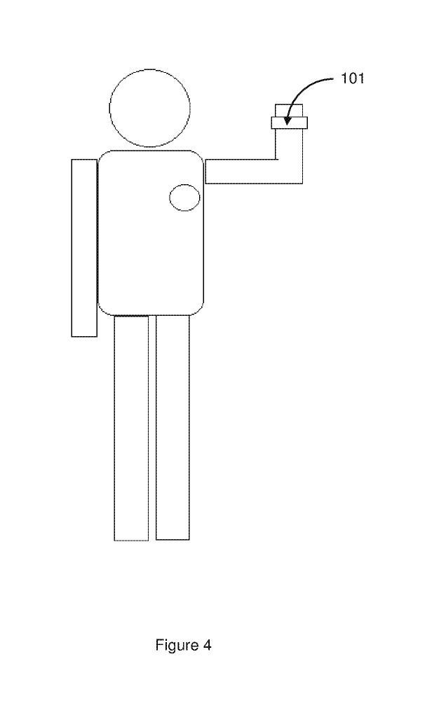

FIG. 4 further illustrates how a person may wear the heart monitor 101 on his wrist as a wrist band. In other embodiments, the heart monitor 101 may be configured to be worn on other parts of the body, such as on the arm in the form of an arm band or on a finger in the form of a ring (not illustrated).

Preferably, a mobile application is installed in the person's mobile phone to collect data from the heart monitor 101, and display the data and an analytical report of his heart condition, as well as forwarding the data to a server for storage or for further processing and retraining of the machine learning algorithm. If an alarm of possibly imminent cardiac arrest is raised, the mobile application is able to display information on the screen of the mobile phone to direct the person to the nearest emergency services or AED (Automatic External Defibrillator) machines. An AED is a device that gives electric shock as therapy to the heart, in order to re-establish normal heart contraction rhythms.

Optionally, the heart monitor 101 is able to issue an alarm over the Internet or telecommunication network to a specific caregiver or an emergency service provider.

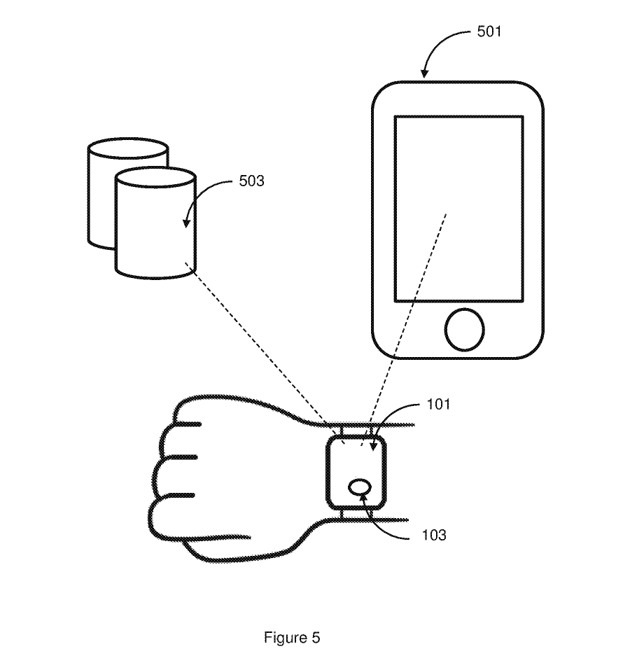

FIG. 5 illustrates that the heart monitor 101 is capable of wireless communication directly with a mobile phone 501 and a server 503. In another embodiment, the heart monitor 101 is part of a smart watch (not illustrated) with its own Internet communication functions and user interactive abilities, bypassing the need for an application in a smart phone.

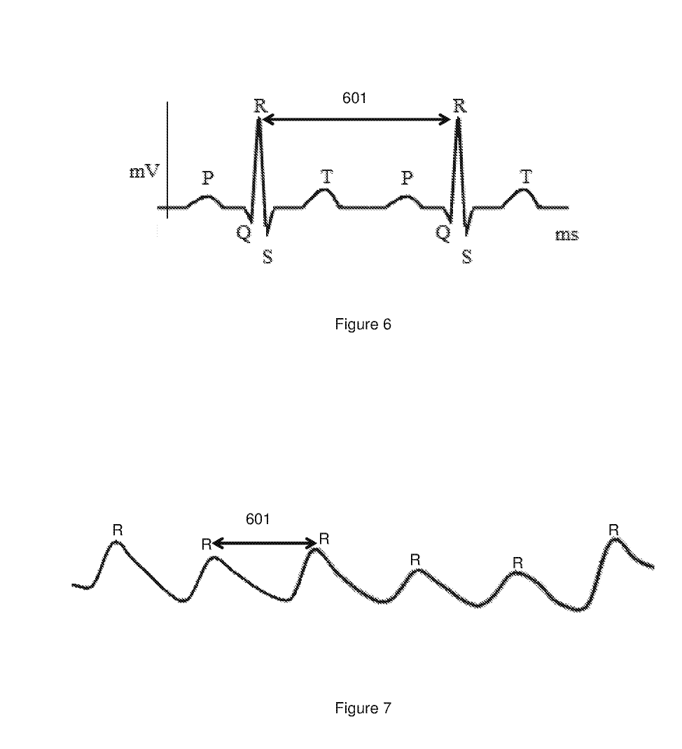

FIG. 6 shows two successive pulses sampled in an ECG. Every ECG pulse has the peaks and troughs labelled PQRST, where P is the point where there is atrial contraction (top heart chamber), S is the point where there is ventricular contraction (bottom heart chamber) and T is the point where there is relaxation. The peak R is the largest peak in each pulse, and is the easiest point by which the interval between two pulses is measured. Therefore, the interval between two pulses is known as the RR interval 601. Sometimes, it is known as the NN interval, meaning “normal to normal” interval.

FIG. 7 shows a chart of heart rhythm obtained by the PPG sensor 309 in the heart monitor 101. The morphology of the pulses obtained by the PPG sensor does not show the same detail as the pulses obtained by ECG. With most currently available, commercial PPG sensors, heart rhythm of a person read by light reflected or rebounded from skin and human tissue does not normally reveal the P and T peaks.

The R peak, however, is easily observed. Therefore, it is possible to measure the RR interval in a person's heart rhythm without using ECG and by using only a PPG sensor.

The algorithm in the heart monitor 101 analyses specific features of the variation in RR intervals as a time series obtained by the PPG sensor 309 to make an assessment of the likelihood of a cardiac arrest. Analysis of trends and changes in the RR interval is termed Heart Rate Variability (HRV) analysis. In contrast, the prior art deemed PPG to be inferior compare to ECG for the purpose of monitoring heart activity. Therefore, the prior art work focused on analysing the morphology of the ECG waveform and rejected the usefulness of HRV analysis for assessing risk of cardiac arrest.

HRV is correlated to the autonomic nervous system of a person. The autonomic nervous system is a part of the nervous system that influences the function of internal organs, and is responsible for control of the bodily functions not consciously directed, such as breathing, the heartbeat, and digestive processes. The autonomic nervous system has two branches: the sympathetic nervous system and the parasympathetic nervous system Before the onset of cardiac arrest, specific activation patterns of the sympathetic and parasympathetic systems should be observable in the variation of the heart rate. The algorithm in the heart monitor 101 looks for these variation patterns in the person's heart rhythm in order to assess the risk of imminent cardiac arrest to give advance warning, i.e. HRV analysis.

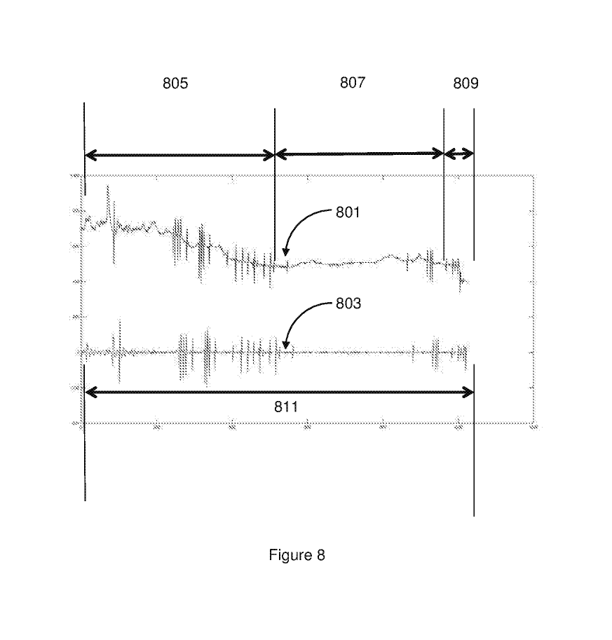

FIG. 8 is a chart obtained by monitoring the heart rhythm of a person for around 10 minutes leading to onset of cardiac arrest. The vertical axis in FIG. 8 is RR interval in milliseconds. The horizontal axis simply represents sampling time. Therefore, the chart shows change in RR interval for every successive R peak and the R peak immediately earlier, i.e. in moving peak pairs. A greater value on the vertical axis indicates a greater time interval between two R peaks, and a lower value indicates a shorter time interval between two R peaks.

Typically, the more uniform the RR intervals, the less variation in the RR intervals. Similarly, the shorter the RR intervals, the less variation in the RR intervals. However, if the heart is functioning normally, the RR interval is not consistent but fluctuates, i.e. the RR interval becomes greater or smaller in an irregular manner.

This is a normal physiological phenomenon. In contrast, heart rate variability is low when a person is about to suffer cardiac arrest.

The top line 801 in FIG. 8 shows the RR interval in the heart rhythm getting progressively shorter as time passes (from the left of the chart to the right), and is marked in three parts. There is a first, leftmost part labelled ‘805’ during which the RR interval gets shorter gradually. There is a second part labelled ‘807’ during which the RR interval is somewhat stable and there is less variation, foreboding imminent cardiac arrest.

The third and rightmost part labelled ‘809’ during which the RR interval gets much shorter suddenly has even less RR interval variation, indicating an increasing heartbeat. The heartbeat in this part which shows that the person is suffering an episode of ventricular tachycardia (VT), which is a form of cardiac arrest.

Accordingly, the gradual reduction of the RR interval in the first part 805 and the low RR interval in the second part 807 are both indicative of the imminent onset of cardiac arrest in the third part 809. Characteristics and features of the variability of the RR interval, i.e. HRV, can be extracted from the first part 805 and the second part 807 and used indicators of whether cardiac arrest in the third part 809 is likely to occur.

As the skilled man knows, VT is an abnormally rapid heartbeat that arises from improper electrical activity in the bottom chambers (ventricles) of the heart. During VT, the ventricles contract in a rapid, unsynchronized way. That is, the ventricles “fibrillate” instead of beat rhythmically at a healthy pace. As a result, the heart may pump little or no blood. This may lead to ventricular fibrillation (VF), sudden cardiac arrest (SCA) or death.

The bottom line 803 in FIG. 8 is extracted from the top line 801 and shows how the top line 801 is interpreted in the prior art. Typically, the low frequency components in the top line 801 are filtered away or ‘de-trended’ to obtain the bottom line 803. The high frequency components are monitored in the bottom line 803 for rapid heartbeat only, or short RR intervals, which is indicative of VT. Thus, no attention is paid to the moving trends of HRV features in the prior art, as the typical analysis has been to observe stationary properties of the heart instead of how the heart rhythm transits from high HRV to low HRV (i.e. from the first part 805 to the second part 807) prior to onset of cardiac arrest. In contrast to the prior art, the present embodiment analyses the moving trend of the underlying dynamics of the heart as seen in the top line 801.

It should be noted that the sudden spikes of RR intervals throughout the chart of FIG. 8 are single problematic and irregular heartbeats, called ectopic beats. These beats are generally removed by signal processing methods prior to analysis of the heart rhythm as they happen in random.

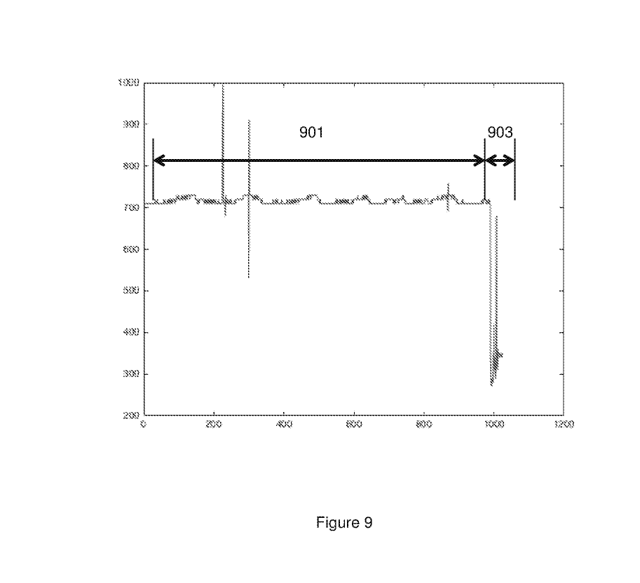

FIG. 9 is another chart having the same axes as the chart of FIG. 8. The leftmost part 901 of the line is where a cardiac arrest has not occurred. A cardiac arrest is captured in the rightmost part 903 of the line, as a sudden drop in the RR interval (vertical axis).

Accordingly, the heart monitor 101 is able to assess the likelihood of onset of VT or VF before it actually occurs by analysing the variability of RR intervals, i.e. the HRV. Specific features are extracted from a one-minute window of RR intervals of the person's heart rhythm in real time to tell whether the heart is functioning normally or if the heart is about to enter into cardiac arrest. Non-exhaustive examples of such features which may be obtained by HRV analysis are listed in Table 1.

TABLE 1
IN TIME DOMAIN

| | |
|---|---|
| 1 | MeanRR—The average period between successive RR intervals. |
| 2 | SDNN—The standard deviation of the period between successive RR |
| | intervals. |
| 3 | RMSSD—Root mean squared differences between successive RR |
| | intervals. |
| 4 | pRR50—The proportion of interval differences between successive |
| | RR intervals having a difference from an earlier RR interval of more |
| | than 50 ms. This will show whether each pulse is having a shorter |
| | period from an earlier pulse. |

IN FREQUENCY DOMAIN

| | |
|---|---|
| 5 | VLF—Power in very low frequency range (0-0.04 Hz) |
| 6 | LF—Power in low frequency range (0.04-0.15 Hz) |
| 7 | HF—Power in high frequency range (0.15-0.4 Hz) |
| 8 | LF/HF—Ratio of the power in low frequency over power in high |
| | frequency. |

FACTORS FOR NON-LINEAR ANALYSIS

| | |
|---|---|
| 9 | SD1 of Poincaré plot |
| | $SD 1 = \frac{1}{\sqrt{2}} \times \sqrt{\frac{var ( {RR}_{n} - {RR}_{n + 1} )}{2}}$ |
| | Where RR is the peak to peak interval of any given pair of successive |
| | peaks. |
| 10 | SD2 of Poincaré plot |
| | $SD 2 = \sqrt{2 \times {SDNN}^{2} - \frac{SD {1}^{2}}{2}}$ |
| 11 | SD1/SD2—Ratio of SD1 over SD2 |
| 12 | ApEn—Approximate Entropy as a measure of chaos |

Typically, after correcting ectopic heart rhythm in the one minute window, four time domain parameters (Mean of the RR intervals, Standard Deviation or SD of the RR intervals, Root Mean Square or RMS of the SD, and pRR50) and three nonlinear parameters of Poincare plot (SD1, SD2, and SD1/SD2, see Table 1) as well as Approximate Entropy (ApEn) were extracted from the RR intervals. Then a Lomb Periodogram was used to obtain the spectral power density curve. The spectral powers in the VLF (very low frequency), LF (low frequency), and HF (high frequency) regions were then calculated. Finally approximate entropy was calculated in the specific time window.

The specific thresholds distinguishing VLF, LF and HF is established only by a machine learning algorithm, that is, the machine learning algorithm is used to find out these thresholds in order to achieve the highest predictive accuracy. The machine learning algorithm is also used to find the thresholds for the other features, and as linear or non-linear combinations of each feature.

Machine learning is a kind of predictive analytics or predictive modelling, and is a study of pattern recognition using artificial intelligence. Typically, machine learning is used to construct algorithms that can learn from and make predictions on data. Such algorithms are built from example data inputs to make data-driven predictions, and are employed when designing and programming explicit algorithms is infeasible. Specific details of machine learning methods are known, the details of which need not be explained here.

The machine learning algorithm in the memory 303 in the heart monitor 101 is preferably an Artificial Neural Network (ANN) algorithm. The features extracted from the one minute window of RR intervals are fed into the ANN to assess the likelihood of cardiac arrest in the near future.

As the skilled man knows, an ANN is a machine learning technique that takes in multiple input parameters to predict specific classes of outcome. An advantage of using machine learning to predict cardiac arrest is that the accuracy, sensitivity and specificity of the algorithm improves as more people use the heart monitor 101 and the amount of historical data grows.

Therefore, to assess the risk of an occurrence of cardiac arrest, the ANN takes in the features in Table 1. The features are supplied in real time just as the heart rhythm is sampled in real time by the PPG sensor 309. If from the features, the ANN algorithm works out that cardiac arrest is likely to occur, the heart monitor 101 raises an alarm.

However, in order for the ANN to be able to make a prediction of cardiac arrest, the ANN first has to be trained to do so. One way of training the ANN is to provide historical data of patients who suffered cardiac arrest in hospitals while having their ECG taken. The features listed in Table 1 are extracted from the RR intervals of these ECG, and fed into the ANN for training. That is, a number of samples of the 5 to 15 minute of heart rhythm leading up to cardiac arrest is obtained from a database of people who have suffered cardiac arrest, and the features of these samples are extracted and used to train the ANN. After the ANN is trained, it can be used to read features extracted from the RR intervals trend of the person wearing the heart monitor 101 to look for signs of cardiac arrest 5 to 15 minutes before onset.

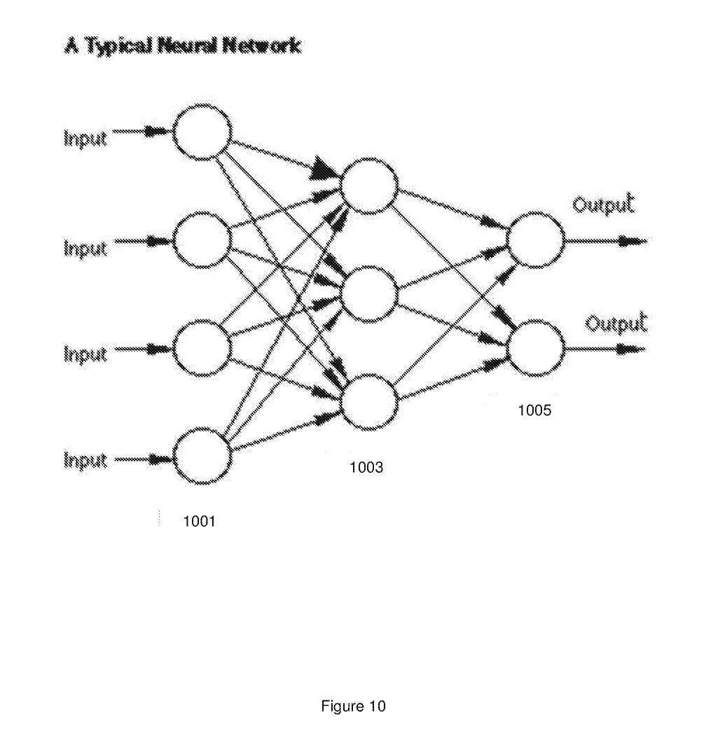

FIG. 10 shows a basic structure or topology of an ANN. The left most column of nodes represents an input layer 1001 into which the features in Table 1 can be fed. The right most column of nodes 1005, two in this example, representing the possible classes of outcome of the features fed into the input layer. In this example, two classes of outcome are VT/VF and ‘normal’. The centre column of nodes 1003 is an over-simplified illustration as there may be more than one centre column. This centre column is called the hidden layer 1003 as the operator of the ANN does not really need to interact with this layer. Each node in the hidden layer 1003 contains an algorithm for assigning a weightage to each feature in order to arrive at the known outcome. Further details of ANN are known to the skilled man and detailed description is not necessary.

In practice, the topology of the actual ANN may be determined experimentally. It has been found that additional hidden layers did not yield better results than a single hidden layer network for the current embodiment, and that a hidden layer of 30 neurons yielded the best results.

Optionally, the features are also extracted from historical RR interval trends of people who did not suffer cardiac arrest and fed into the ANN with an indication that the class of outcome is ‘normal’. This will train the ANN to recognise patterns in the features which point to a low likelihood of occurrence of cardiac arrest.

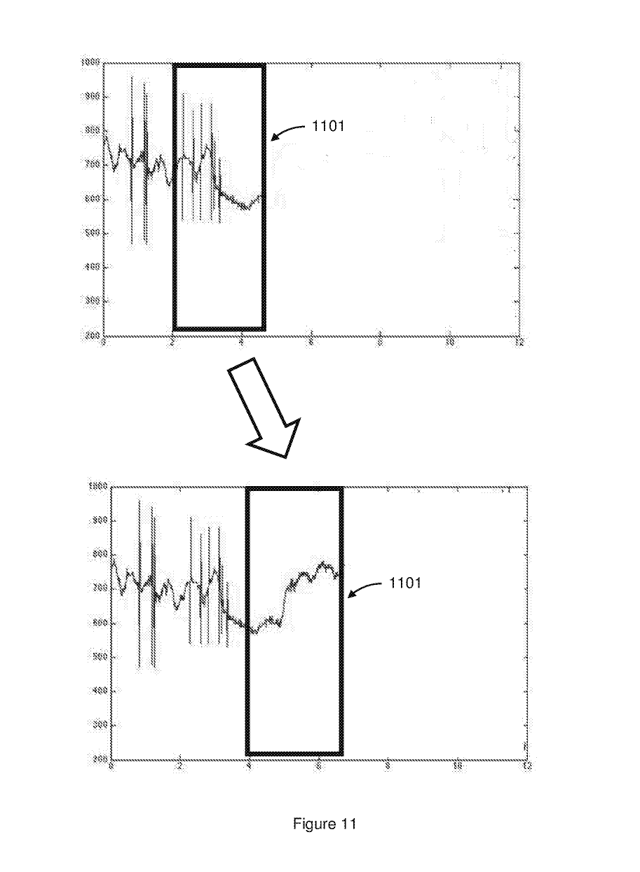

FIG. 11 shows how a one minute moving window 1101 is applied to a chart of RR intervals. The top chart is the same as the bottom chart except that the top chart shows an earlier point in time while the bottom chart shows a later point in time. The chart of RR intervals is updated in real time as the PPG sensor reads the person's heart rhythm. The one minute window 1101 ‘moves’ along to the latest RR interval, as shown moving from the position in the top chart to the position in the bottom chart.

Preferably, the heart sensor 101 also contains a noise titration algorithm as described in US20140213919 to extract non-linear signals more robustly from noisy signals, or contains other algorithm giving similar noise reduction output.

The heart monitor 101 preferably comprises an accelerometer 313 (FIG. 3) for detecting movements of the person wearing it. This would allow the motion artefacts of the PPG signal due to the movement of the person to be cancelled out. That is, by taking account of the readings from the accelerometer 313 as the heart rhythm is sampled, the heart monitor 101 can perform noise cancellation to remove the effect of the person's movements. In the event that the person's movements affect the reading of the heart rhythm too severely and noise cancellation is not possible, the heart monitor 101 suspends the HRV analysis in order to prevent false alarms. Typically, both the accelerometer 313 and the skin impedance sensor 315 help detect mis-positioning of the heart monitor 101 such that an alert can be issued to the person via the mobile phone application to reposition the heart monitor 101, or by a haptic signal in a specific rhythm issued by the heart monitor 101.

One advantage of the embodiment is that, even though the heart monitor 101 is already deployed on the person wearing it, the ANN can still be trained further. The historical chart of the RR intervals of any person who suffers cardiac arrest while wearing the heart monitor 101 can be fed into the ANN to further train the ANN so that the ANN can assess the risk of cardiac arrest more accurately. Therefore, the heart monitor 101 increases in the accuracy of its prediction as it is used by more people and over time.

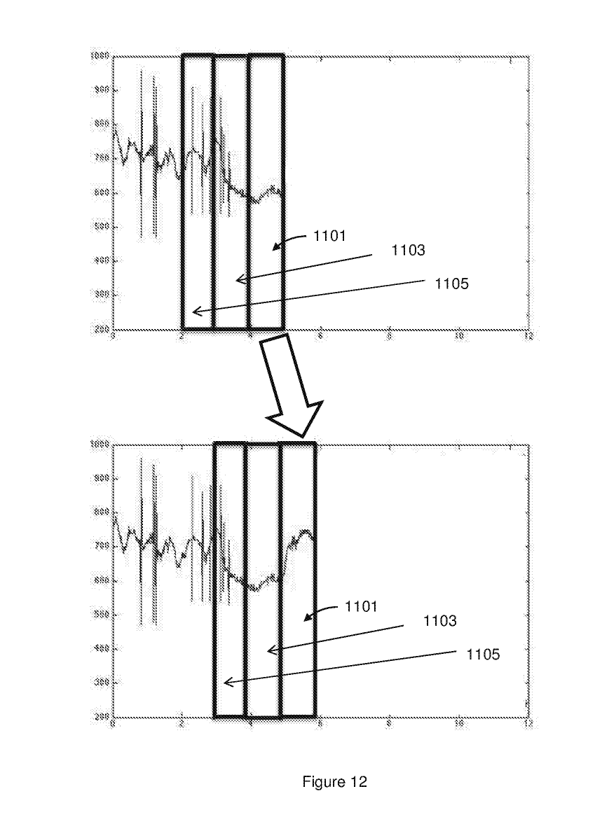

In a variation of the embodiment, the heart monitor 101 takes in data not only from one moving window but a succession of three windows, 1101, 1103, 1105, as shown in FIG. 12. Each window has the same one minute duration but samples different parts of the chart of the RR intervals. The data obtained from the three windows, 1101, 1103, 1105 are analysed concurrently by the ANN. Accordingly, the number of input nodes to the ANN for both the ANN training and prediction is now three times, i.e. thirty six, instead of twelve. The number of output nodes remains the same, as there are only two outcomes in this embodiment, VT/VF or normal.

In other words, each window provides a period of heart rhythm to be subjected to concurrent analysis with the periods of heart rhythm observed in the other windows. The two leftmost windows in FIG. 12 observes each a recent period of the heart rhythm, i.e. somewhat historical, while the right most window observes the most current period of the heart rhythm. Typically, the windows, 1101, 1103, 1105 do not overlap in order not to duplicate input into the ANN.

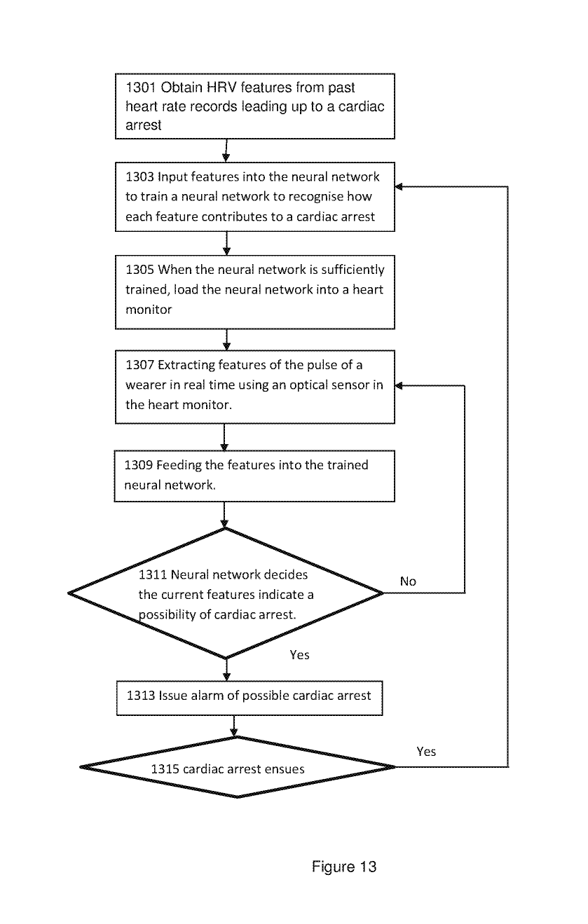

FIG. 13 is a flowchart showing an overall scheme for implementing the embodiment. In the beginning, the ANN is trained. An HRV analysis is performed by extracting the features shown in Table 1 from the historical records of people who were having their ECG taken prior to them suffering a cardiac arrest, at step 1301. The extracted features and the known outcome of cardiac arrest are fed into the ANN to train the ANN to recognise how a linear and nonlinear combination of each feature is predictive of cardiac arrest, at step 1303.

As the heart monitor 101 is battery powered and has limited processing power and memory capacity, it is less expedient for the heart monitor 101 to perform the ANN training itself. Therefore, the ANN is preferably trained in a central computer or a server 503. When the ANN is considered sufficiently trained, the model parameters of the ANN is broadcasted and downloaded into all the heart monitors 101 of the embodiment wirelessly, at step 1305, through mobile Internet and Bluetooth, and via the mobile phone application. By only using an already trained ANN in the heart monitor 101, the heart monitor 101 needs less processing power and memory, and hence allowing the battery 307 to last longer.

In use, each heart monitor 101 monitors the heart rhythm of its wearer continuously by reading the pulse of the wearer via the PPG sensor. The RR interval of each heart beat is obtained in real time, and the features as shown in Table 1 is extracted from a one minute window of the RR interval trend, at step 1307, in real time. The latest features are fed into the trained ANN continuously, at step 1309. Whenever the ANN detects a possibility of cardiac arrest from the features, at step 1311, the heart monitor 101 issues an alarm, at step 1313.

As long as the ANN does not detect a possibility of cardiac arrest, the ANN loops back to step 1307 to continue monitoring the latest RR intervals of the heart rhythm for signs of cardiac arrest.

Typically, there will be many people wearing a heart monitor 101. If anyone wearing a heart monitor 101 suffers a cardiac arrest, the historical features of the RR intervals of that person are taken and fed into a copy of the ANN in the server 503 to further train the copy of the ANN, at step 1315. When the copy of ANN is re-trained, the copy of the ANN is downloaded into all the heart monitors 101, repeating step 1303, to upgrade their predictive accuracy.

Currently, actual records of people's heart rhythm leading up to cardiac arrest are available mostly in about the 5 to 15 minutes prior to onset. However, as the heart monitor 101 is worn by more people throughout the day, the heart monitor 101 is able to collect data on heart rhythm leading up to cardiac arrest even at 30 minutes or an hour prior to onset, which can be used to re-train the ANN to recognise signs of cardiac arrest as advance in time as 30 minutes or an hour.

In another embodiment, both the training and the application of the ANN are conducted in the server 503. In this case, the heart monitor 101 is simply a data-gathering device and the heart rhythm of the person is uploaded to the server 503 in real time for HRV analysis to be conducted and the likelihood of cardiac arrest to be predicted. If cardiac arrest is likely to occur, the server 503 informs the heart monitor 101 wirelessly to raise an alarm.

Accordingly, the embodiments described provides the possibility of a life-saving early warning heart monitor 101 that provides round the clock, daily heart condition monitoring. Any underlying heart problems that the person might have may be detected in advance of an onset of cardiac arrest and a suitable alarm raised. Any irregularities in a person's heart rhythm will trigger a warning system to send out alarms to the person's family members, neighbours and emergency services. This enables faster medical attention, substantially increasing the chance of survival.

Also, the described embodiments provides the possibility to differentiate between the heart rhythm of a healthy heart and one with a heart disease, enabling users to be notified of underlying hidden heart conditions that they were not aware of.

The heart monitor 101 is therefore a wearable device 101 suitable for assessing the likelihood of onset of cardiac arrest, comprising: a wearable configuration for being worn by a body part, a light source 201 configured for illuminating the body part, an optical sensor 203 configured to detect light rebounded from the body part, wherein the heart rhythm of a person wearing the wearable device 101 is detected by the optical sensor 203 from the pulsation in intensity of the rebounded light, and the wearable device 101 is capable of subjecting the heart rhythm to analysis and issuing an alarm if the heart rhythm comprises patterns pre-determined as preceding onset of cardiac arrest.

The heart monitor 101 therefore provides a method for assessing the likelihood of the onset of cardiac arrest comprising the steps of: providing a light source 201 for illuminating a body part of a person, detecting from the pulsation in intensity of the rebounded light rebounded from the body part to detect the heart rhythm of the person, subjecting the heart rhythm to analysis, and raising an alarm if the analysis determines that the heart rhythm comprises patterns pre-determined as preceding onset of cardiac arrest.

While there has been described in the foregoing description preferred embodiments of the present invention, it will be understood by those skilled in the technology concerned that many variations or modifications in details of design, construction or operation may be made without departing from the scope of the present invention as claimed.

For example, although a neural network algorithm is mentioned here, other manner of analysing multivariate factors for a result may be used. For example, Support Vector Machine, K-Nearest Neighbour, or Singular Vector Decomposition, were the few different algorithms that may be used instead of an ANN. Also, the ANN has been described to analyse the RR interval, or heart rate variability observed by a PPG. However, the skilled man will understand that any similar algorithm will also work on RR interval obtained by ECG.

Although the embodiments were described having an ANN of two possible classes of outcomes, there may be as many possible classes as the situation require. For example, in yet another embodiment, there may be three classes of outcome from the ANN, such as VT, VF, or Normal. This allows the ANN prediction to be more refined and specific.

Where server is mentioned herein, the skilled man understands it includes a cloud server.

Although it is described that the RR intervals were analysed in the form of charts and charts, the skilled man understand that this is just a manner of presentation and the RR intervals may be treated as data in a spread sheet or tables and so on, without actually presenting the data as charts of charts.

Although it has been described that RR intervals are intervals between successive pulses, it is possible to take the interval between any consistent number of pulses, such as intervals between every first and third pulse, or any other pre-determined number of pulses.

Although it has been described that RR intervals are analysed in HRV analysis, in some embodiments, any other aspects of the heart rhythm may be analysed instead, such as pulse intensity, where the heart rhythm is obtained by the optical sensor 203.

Where a person is referred to in the description, it is possible that a reference is made to an animal as well.

1. A wearable device suitable for assessing the likelihood of onset of cardiac arrest, comprising:
a wearable configuration for being worn by a body part;
a light source configured for illuminating the body part;
an optical sensor configured to detect light rebounded from the body part;
wherein the heart rhythm of a person wearing the wearable device is detected by the optical sensor from pulsation in intensity of the rebounded light; and
the wearable device is capable of subjecting the heart rhythm to analysis and issuing an alarm if the heart rhythm comprises patterns pre-determined as preceding onset of cardiac arrest.

2. A wearable device suitable for assessing the likelihood of onset of cardiac arrest, as claimed in claim 1, wherein the wearable device is capable of subjecting the heart rhythm to analysis by a machine learning algorithm.

3. A wearable device suitable for assessing the likelihood of onset of cardiac arrest, as claimed in claim 2, wherein the machine learning algorithm is an artificial neural network.

4. A wearable device suitable for assessing the likelihood of onset of cardiac arrest, as claimed in claim 2, wherein the analysis by the machine learning algorithm is made on heart rate variation observed from the heart rhythm.

5. A wearable device suitable for monitoring the heart rhythm of a person, as claimed in claim 1, further comprising an accelerometer configured to detect movements of the person; wherein the analysis of the heart rhythm include cancellation of the effects from movements of the person on the heart rhythm detected by the optical sensor.

6. A wearable device suitable for monitoring the heart rhythm of a person, as claimed in claim 1, further comprising a skin impedance sensor; the skin impedance sensor positioned on the wearable device such that impedance measured by the skin impedance sensor is indicative of contact between the optical sensor and the skin of the person.

7. A wearable device suitable for monitoring the heart rhythm of a person, as claimed in claim 1, wherein the wearable device is configured as a wristband.

8. A wearable device suitable for assessing the likelihood of onset of cardiac arrest, as claimed in claim 3, wherein the artificial neural network is trained using records of heart rhythm of at least the 15 minutes leading up to cardiac arrest.

9. A wearable device suitable for assessing the likelihood of onset of cardiac arrest, as claimed in claim 3, wherein the artificial neural network is trained using records of heart rhythm of at least the 30 minutes leading up to cardiac arrest.

10. A method for assessing the likelihood of the onset of cardiac arrest comprising the steps of:
providing a light source for illuminating a body part of a person;
detecting from the pulsation in intensity of the rebounded light rebounded from the body part to obtain the heart rhythm of the person;
subjecting the heart rhythm to analysis; and
raising an alarm if the analysis determines that the heart rhythm comprises patterns pre-determined as preceding onset of cardiac arrest.

11. A method for assessing the likelihood of the onset of cardiac arrest as claimed in claim 10, wherein the step of subjecting the heart rhythm to analysis is to apply an algorithm to analyse the heart rhythm; and the algorithm is a machine learning algorithm.

12. A method for assessing the likelihood of the onset of cardiac arrest as claimed in claim 11, wherein the machine learning algorithm is an artificial neural network.

13. A method for assessing the likelihood of the onset of cardiac arrest as claimed in claim 10, wherein the analysis is made on heart rate variation observed from the heart rhythm.

14. A method for assessing the likelihood of the onset of cardiac arrest as claimed in claim 10, wherein the heart rhythm is obtained from a pre-determined number of windows of time; each window providing a period of heart rhythm to be subjected to concurrent analysis with the periods of heart rhythm observed in the other windows.

15. A method for assessing the likelihood of the onset of cardiac arrest as claimed in claim 12, wherein the artificial neural network is trained using records of heart rhythm of at least the 15 minutes leading up to cardiac arrest.

16. A method for assessing the likelihood of the onset of cardiac arrest as claimed in claim 12, wherein the artificial neural network is trained using records of heart rhythm of at least the 30 minutes leading up to cardiac arrest.
